(19) United States
(12) Patent Application Publication
(10) Pub. No.: US 2011/0004048 A1
(43) Pub. Date: Jan. 6, 2011

(54) THERAPY DEVICES AND DOMESTIC/COMMERCIAL THERAPY SYSTEM

(76) Inventor: Henry Brunelle, Cap-Rouge (CA)

Correspondence Address: OGILVY RENAULT LLP, 1, Place Ville Marie, SUITE 2500, MONTREAL, QC, H3B 1R1, CA

(21) Appl. No.: 12/497,562

(22) Filed: Jul. 3, 2009

(51) Int. Cl. A61M 21/00

(52) U.S. Cl. 600/28; 600/27

(57) ABSTRACT

A therapy table/chair device having integrated therewith a head rest support and body supports and wherein the head rest support provides face exposure to a sensory generating housing through an opening in the head rest support. The sensory generating housing has at least one or a combination of facial applied therapies including lumino therapy, audio therapy, aroma therapy, ozone, steam and oxygen injectors with or without scented essences. The body supports are part of a massage table and are provided with at least one or a combination of vibration, magnetic field and heat therapy. A controller controls all of these therapies and an interface permits the selective operation thereof. The therapy table/chair is also integratable in a personalized therapeutic treatment home spa which is interfaced with a commercially operated spa offering therapeutic services to the household spa. Communication between the commercially operated spa and the household spa is effected through an internet link and computers.

### TECHNICAL FIELD

The present invention relates to therapy devices adapted to provide to a user person facial/mentally applied therapies as well as body applied therapies. The massage therapy devices are also integratable in a personalized therapeutic treatment system which is comprised of a commercially operated spa offering therapeutic services and a household spa having one or more therapeutic devices.

### BACKGROUND ART

It is known in the prior art to provide massage therapy tables and chairs and incorporating therein devices to provide to the user person various forms of therapy such as virtual reality experiences, aroma therapy, ozone or oxygen therapy, heat therapy and other such therapies that may be incorporated in such massage devices. With reference to U.S. Pat. Nos. 6,273,865 and 7,080,420, examples of such devices are therein described. However, such devices all have limited use and in most cases are limited to massage therapy, thus requiring the presence of a masseur person.

### SUMMARY OF INVENTION

It is a feature of the present invention to provide therapy devices which are an improvement over the devices known in the art and which are provided with selectable sensory generating means to transmit to the face, brain and various parts of the body of the user person therapies.

Another feature is to provide a massage table having integrated therewith various therapeutic means and providing for a massage by a masseur person.

Another feature is to provide a transportable massage table which is adapted for support on a free standing bathtub and wherein therapeutic means are secured on the underside of the massage table to provide one or more therapeutic treatment to a person lying in the bathtub under the massage table.

Another feature of the present invention is to provide a personalized therapeutic treatment system which is comprised of a commercially operated spa offering therapeutic services and a household spa having one or more therapeutic treatment devices with both the commercial and household spas communicating through an internet link whereby the household spa may benefit from a full line of therapeutic services at home.

Another feature of the present invention is to provide a personalized therapeutic treatment system comprising a commercially operated spa offering therapeutic services, and a household spa having one or more therapy table/chair constructed in accordance with the present invention.

According to the above features, from a broad aspect, the present invention provides a therapy device adapted to support the body or part of the body of a user person. The device comprises body supports secured to a main frame and positioned for supporting at least portions of the body of the user person. A head rest support is adapted to support a peripheral portion about the face of the user person while permitting face exposure to a sensory generating housing. The sensory generating housing has at least one of or a combination of facial applied therapies including a lumino therapeutic means, an audio therapy device, an aroma therapeutic means, an ozone or oxygen injector, and scented essences. The body supports have at least one of or a combination of body applied therapies including a vibration generating means, a controllable magnetic field generator and heating means. A controller device has an interface with inputting means for selectively actuating each or all or a combination of the facial and body applied therapies. The controller has a computer and memory for storing executable programs actuable by instructions generated by the inputting means.

According to a further broad aspect of the present invention there is provided a therapy device adapted to support the body or part of the body of a user person. The device comprises body supports secured to a support frame and positioned for supporting at least portions of the body of the user person. The body supports have at least one of or a combination of body applied therapies including a vibration generating means, a controllable magnetic field generator and heating means. A controller device has an interface with inputting means for selectively actuating one or all or a combination of the body applied therapies. The controller has a computer and memory for storing executable programs actuable by instructions generated by the inputting means. The body supports are constituted by a seat or a seat portion of a transport vehicle.

According to a further broad aspect of the present invention there is provided a therapy device adapted to support a facial part of the body of a user person. The device comprises a head rest support adapted to support a peripheral portion about the face of the user person while permitting exposure to a sensory generating housing having at least one or a combination of facial applied therapies including lumino-therapeutic means, an audio therapy device, an aroma therapeutic means; an ozone, oxygen or water vapor injector, and scented essences. A controller device has an interface with inputting means for selectively actuating one or all or a combination of the facial applied therapies. The controller has a computer and memory for storing executable programs actuable by instructions generated by the inputting means. The head rest support is secured to support means disposed forwardly of a seat of a transport vehicle.

According to a further broad aspect of the present invention there is provided a personalized therapeutic treatment system which comprises a commercially operated spa offering therapeutic services, and a household spa having one or more therapeutic treatment devices. An internet link is provided for communicating information between the commercially operated spa and the household spa and relating to one or more therapy treatment programs assigned to one or more domestic user persons of the household spa using the treatment devices.

### BRIEF DESCRIPTION OF DRAWINGS

A preferred embodiment of the present invention will now be described with reference to the accompanying drawings in which:

FIG. 1 is a simplified schematic view of a massage therapy table incorporating therein a plurality of facial applied therapies as well as body applied therapies;

FIG. 2A is a schematic side view of a massage therapy chair constructed in accordance with the present invention and providing a plurality of facial applied as well as body applied therapies;

FIG. 2B is a front view of FIG. 2A;

FIG. 3 is an enlarged view showing the basic construction of the head rest support pad and its relation to the sensory generating housing;

FIG. 4 is a perspective view illustrating a modification of the massage table herein adapted or mounting over a bathtub;

FIGS. 5A and 5B are perspective views showing the massage table deposed on a bathtub;

FIG. 6 is a fragmented view showing the position of a flexible heating membrane held captive between a foam layer and covering material of the massage table;

FIG. 7 is a simplified cross-section view showing a user person lying in a bathtub, under the massage table, and undergoing therapeutic treatments; and

FIG. 8 is a simplified block diagram illustrating a personalized therapeutic treatment system comprised of a domestic spa interfaced with a commercial spa through an internet link.

### DESCRIPTION OF PREFERRED EMBODIMENTS

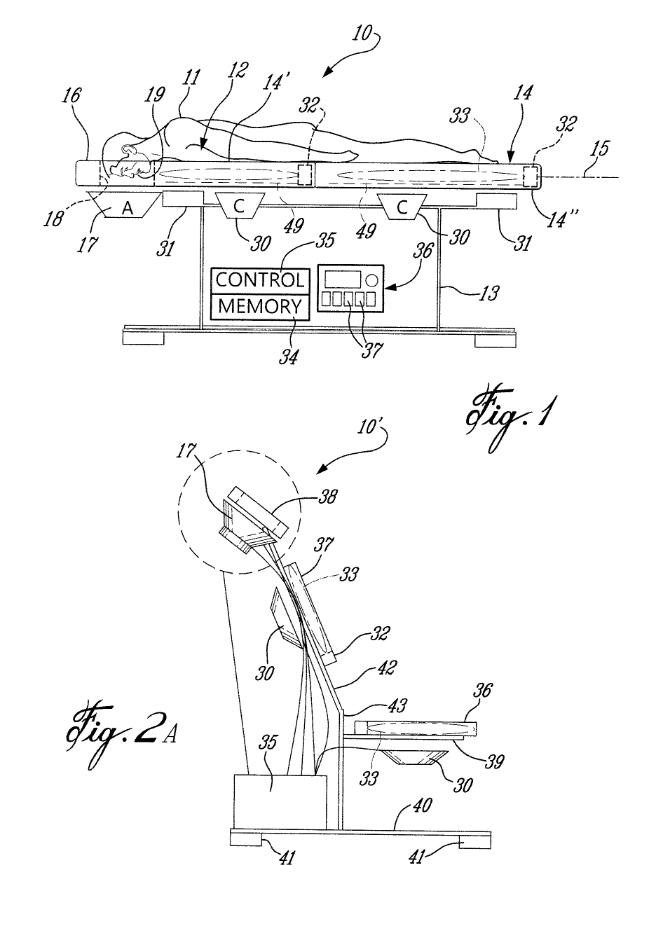

Referring now to the drawings and more particularly to FIG. 1, there is shown generally at 10 an example of a massage therapy table constructed in accordance with the present invention and adapted to support the body 11 of a user person 12. The massage therapy table 10 comprises a main frame 13 having a framework suitable for supporting the table platform with body support pads 14 along a horizontal top plane 15 thereof. As hereinshown there is provided an upper body portion support pad 14′ and a lower body portion support pad 14″. These support pads may be interconnected by hinges to articulate them.

Each of the body portion support pads 14′ and 14″ are provided with body therapy devices, operated independently from one another, to generate a therapeutic treatment to the user person 12 lying on the body support pads. The upper body portion support pad 14′ is provided with a head rest section 16 at a free end thereof. A sensory generating housing 17 is secured under the upper body portion support pad 14′ and aligned with an opening 18 of the head rest section 16 to permit face exposure thereto as herein shown and wherein the face 19 of the user person is disposed within the opening 18 with the head being supported by a peripheral pad portion about the opening.

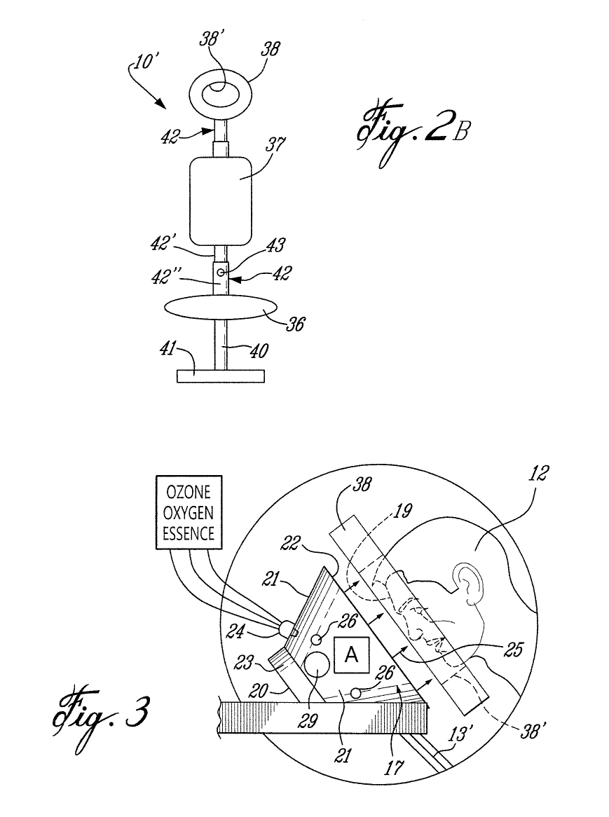

With additional reference now to FIG. 3, it can be seen that the sensory generating housing 17 is secured to a frame member 13′ in a manner to be removably connected thereto and disposed in closed space proximity to the face 19 of the user person 12. It may also be telescopically supported for ease of adjustment. The sensory generating housing 17 has a closed bottom end 20, surrounding side walls 21 and an open top end 22. In one embodiment the closed bottom end 20 may have an image generating screen device secured therein to provide visual therapy to the eyes of the user person. A skin contact virtual visual and sound experience can also be provided through the screen as well as light therapy. Ozone, oxygen, vapor and/or scented essence injectors 24 are also secured to the side wall 21 or bottom end wall 20 of the housing 17 to generate ozone and oxygen, whether scented or not, to the face of the user person as indicated by arrows 25. Such could provide, for example, the sensory experience of a forest or ocean waves transporting the sensory experience of the user person to a relaxed environment.

The injectors 24 may also be pulsating injectors and these may be operated in a sequential mode as programmed in a controller 35 which will be described later. A plurality of light emitting diodes 26 are secured to the side walls 21 of the housing 17 to provide chromo therapy. One or more speakers 29 may also be connected to the side walls 21 of the sensory generating housing 17 to generate sound or music appropriate to a therapy.

As shown in FIG. 1, the body support pads 14′ and 14″ have at least one or a combination of body applied therapies which include a vibration generating means in the form of vibrating elements, herein frequency operated cones 30, disposed to impart vibrations to the pads 14′ and 14″ or frame elements 31 supporting the pads. Controllable magnetic field generators 32 equipped with coils or other form of conductors, as will be described later, are also secured within the foam inside pads 14′ and 14″ to generate an oriented magnetic field 33 within the immediate surface area of the pads to provide electromagnetic therapy to the user person's body or parts of the body, such as the legs or arms of the user person. The intensity modulation and frequency of the magnetic field is controlled by an executable program which is stored in the memory 34 of a controller device 35. The controller device has an interface module 36, or integrated therein, and provided with touch switches 37 to select desired ones of the various facial or body applied therapies. Several executable programs are stored in the memory 34 of the controller 35. Electrical heating wires 49 and 49′ are also incorporated in the upper body support pad 14 and lower body support pad 14″. Solid membrane infrared panels may also be secured to ports of the pads to generate heat to the body or parts thereof. The therapy devices of the present invention can be used by the user person without assistance, if a masseur is not required. Well known massaging devices may also be incorporated in the pads to effect different type of massages and accordingly a masseur person is not required.

With reference now to FIGS. 2A and 2B, there is shown a massage therapy chair 10′ also constructed in accordance with the present invention. The massage therapy chair 10 is provided with a seat support pad 36, and abdominal support pad 37 and a head rest support pad 38. The seat support pad 36 is secured to a horizontal seat frame portion 39 of the main frame 40 which is provided with legs 41. An inclined abdominal frame member 42 extends angularly upwards and outwards from an edge section 43 of the horizontal seat frame portion 39 with the abdominal pad 37 secured thereto and spaced from the seat pad 36 and the head rest support pad 38. The head rest support pad 38 is secured to a free end of the inclined abdominal frame member 42 and spaced from a top end of the abdominal frame.

As shown in FIG. 2B, the abdominal frame member 42 is provided with telescopic sections 42′ and 42″ in both the areas of the frame below and above the abdominal support pad 37 whereby to adjust the chair to user persons of different stature. A connecting knob 43 provides quick and easy adjustment and interconnection of the telescopic frame members 42′ and 42″.

As shown in FIG. 2A, the seat support pad 36 and abdominal support pad 37 are also provided with electromagnetic field generators 33. They are also provided with vibration devices 30 as previously described. Similarly, the head rest support pad is provided with the housing 17 as previously described to selectively provide all of the therapies associated therewith, as shown in FIG. 3 and described above. As shown in FIG. 2A, the head support pad is an annular pad having an opening 38′ therein for receiving the face 19 of the user person.

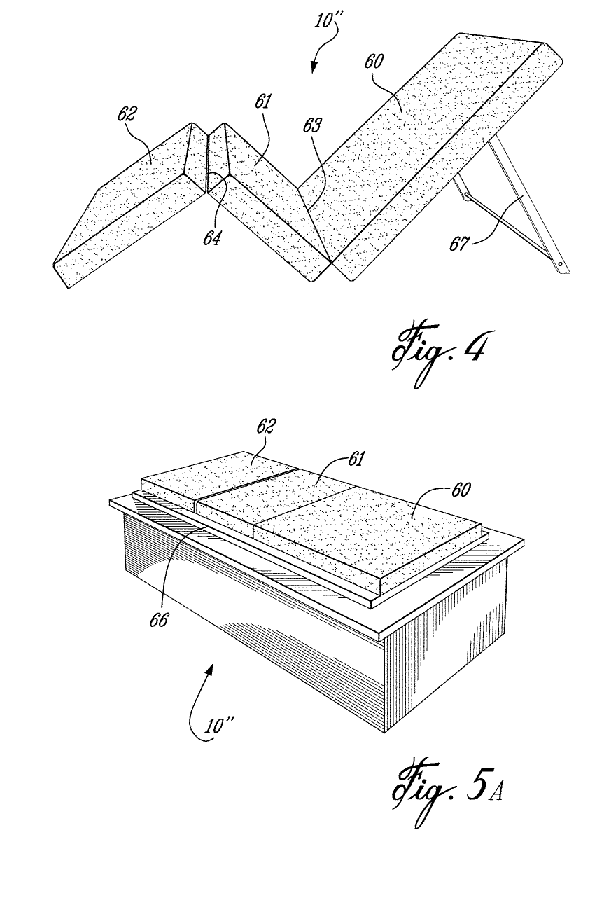

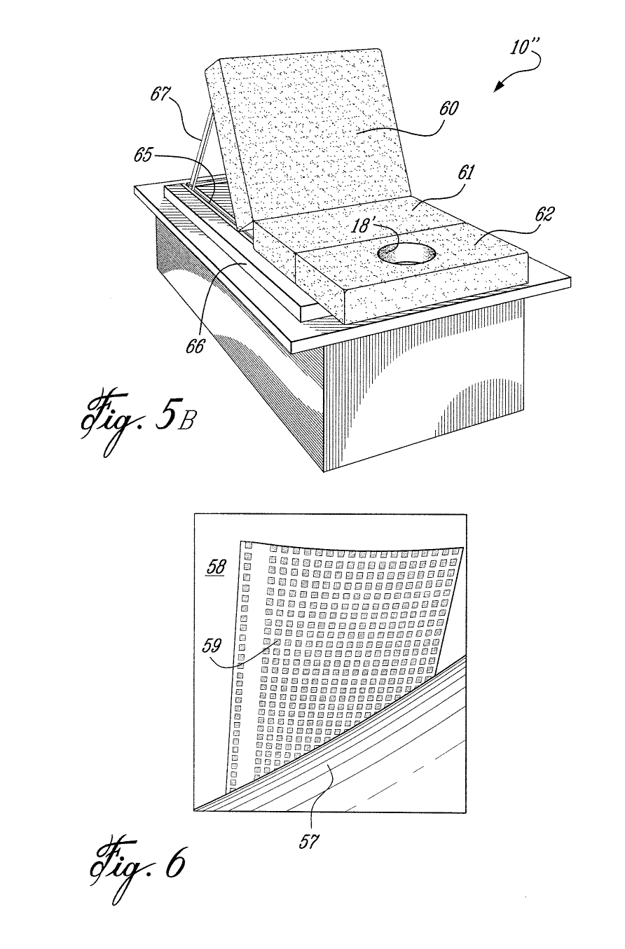

Referring now to FIGS. 4 to 7, there is shown a further embodiment of the therapy table. As hereinshown, the therapy table 10″ is provided by three rigid foam pad panels 60, 61 and 62 interconnected together by hinges 63 and 64. An adjustable frame 65, see FIG. 5B, is secured under the panels or provided separately to mount the pads onto an existing bathtub 66, herein a free standing bathtub. It could also be mounted on a recessed bathtub if a masseur person is not required, such as when massaging devices are integrated in the panels and automatically operated by the controller 35. The hinges provide for the panels to be articulated and an adjustable backrest support 67 maintains the panel 60 at an angle when required. As shown in FIG. 6, a flexible heating membrane 59 is held captive between the foam sheet 58 and the covering material 57.

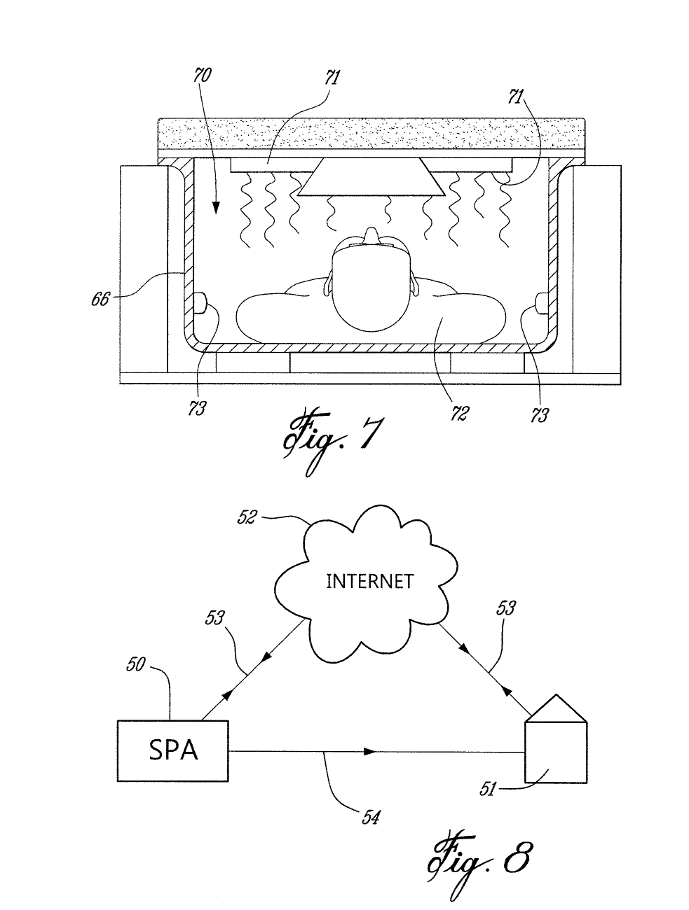

As shown in FIG. 5B, one of the panels, herein panel 62, may be provided with a face receiving opening 18′ under which is detachably secured the sensory generating housing 17. The bathtub 66 thus provides the support for the massage table 10″. It also provides an enclosure 70 thereunder formed by the bathtub. This enclosure 70 is illustrated in FIG. 7.

The enclosure 70 forms a therapeutic chamber in which a person can receive some of the sensory therapies described previously. As shown in FIG. 7, the undersides of the pads are fitted with infrared radiating panels 71 to provide a heat sensation to the body of the user person 72 lying in the bathtub 66. The sensory generating housing 17, which is detachably securable, may also be reversibly attached to generate the sensory therapies previously described. The bathtub 66 may also be a therapeutic bathtub equipped with massaging jet 73 and water may also be provided at a safe level to provide further therapeutic treatment. Accordingly, the massage table 10″ provides for further therapeutic use in a house spa where the bathtub can be used as a support and treatment chamber.

The therapy table 10 and chair 10′ have also been conceived for domestic use and these therapeutic treatment devices can be incorporated in a personalized therapeutic treatment system as shown in FIG. 8. The system as shown in FIG. 8 integrates a commercially operated spa 50, offering therapeutic services, to a household spa 51 having one or more therapeutic treatment devices such as the therapeutic table 10, 10″ and chair 10′ of the present invention or other therapeutic treatment devices. Through the internet 52, a computer link 53 is established between the household spa 51 and the commercially operated spa 50. This link provides for communication of information, through the use of PCs 55, relating to one or more therapy treatment programs assigned to one or more domestic user person of the household spas 51, only one hereinshown. The one or more therapy treatment programs include massage therapy programs which are assigned by a coach person of the commercially operated spa in communication with the person of the household spa through the internet. A masseur person may visit the household spa 51 on a periodic basis, as required, depending on the treatment program, and this is represented by the link 54 between the commercially operated spa 50 and the household spa 51. Accordingly, all forms of therapeutic treatments and services can be offered to a person at home, having its own custom spa, without having to visit a commercially-operated spa 50 on a regular basis. Accordingly, this method offers health improving services for the well being of a person desirable of having all of the services at home. Through the internet link 53 the domestic user person would also provide information pertaining to its regular customized treatment programs to a masseur person or other coach person at the commercially operated spa 50 to effect a follow-up and/or the coach to monitoring of results. The controller 35 may have a program to store data pertaining to the treatment program and which data is made available to the commercial spa via the internet link.

The household spa 51 may be a bathroom of the household or a room dedicated to contain various therapeutic appliances such as sauna or steam shower enclosures, hydromassaging bathtubs and visual and other virtual therapy generating devices incorporated in the special room. See our co-pending U.S. application Ser. No. 12/185,163, filed on Aug. 4, 2008 and entitled “Wall Integrated Multisensory Therapy Device”, the contents of which are incorporated herein with the embodiment of FIG. 8.

The commercial generating spa 50 would also offer through the internet link, therapeutic products personalized to the domestic user persons of all household spa clients connected to the commercial spa through the internet. Internet accessible program forms are also communicated to the user persons at the household spas via the internet and provide a means of communicating information on ongoing treatment programs of each home spa user whereby a coach or an assigned qualified person, at the commercial spa, can evaluate the performance of the user persons and modify the treatment program if necessary. Through the internet, general exercising and nutritional information can also be provided for the benefit of all domestic user person clients of the commercial spa. The therapeutic service of the commercial spa includes visits to the home spas to provide therapeutic products, nutritional information, and exercising classes personalized to the domestic user person as well as courses and techniques relating to the therapeutic treatments and products.

It is within the ambit of the present invention to cover any other obvious modifications of the preferred embodiments described herein provided such modifications fall within the scope of the appended claims. For example, the sensory generating housing 17 may be connected directly to the head rest section 16 of the pad 14′ of the table 10 or the head rest support pad 38 of the chair 10′ by such detachable means as Velcro, etc. Also the vibration imparting means or diaphragms 30 may be substituted by pizocrystal frequency generators. Further, in order to humidify the face 19 of the user person 12, small jets of steam may also be connected to the sensory generating housing 17. Negative ions can also be injected into the sensory generating housing 17. The personalized therapeutic treatment system as above-described is a means to transform a bathroom or dedicated room in a household where therapeutic treatments can be given and a follow-up on the treatments provided, such as with a coach at a gym, without the domestic user person having to visit a commercial spa.

The therapeutic treatment devices as above-described may also be incorporated in the seats of transport vehicles such as cars, buses, trains, ships, etc., whereby to provide relaxing and beneficial therapies to traveling persons, particularly for long voyages, such as long flights. Accordingly, the table therapeutic devices can be integrated in seat and backrest cushions. The sensory generating housing 17 could be secured to the rear of a backrest whereby the traveler person can receive these therapeutic treatments as well or optionally. Such would greatly reduce the effects of jetlag, stress, fatigue and other discomforts associated with travel.

I claim:

1. A therapy device adapted to support the body or part of the body of a user person, said device comprising body supports secured to a main frame and positioned for supporting at least portions of the body of the user person, and a head rest support adapted to support a peripheral portion about the face of the user person while permitting face exposure to a sensory generating housing; said sensory generating housing having at least one or a combination of facial applied therapies including a lumino therapeutic means, an audio therapy device, an aroma therapeutic means, an ozone, oxygen or water vapor injector, and scented essences; said body supports having at least one of or a combination of body applied therapies including a vibration generating means, a controllable magnetic field generator and heating means; and a controller device having an interface with inputting means for selectively actuating one or all or a combination of said facial and body applied therapies, said controller having a computer and memory for storing executable programs actuable by instructions generated by said inputting means.

2. A therapy device as claimed in claim 1 wherein said therapy device is a massage table.

3. A therapy device as claimed in claim 1 wherein said therapy device is a massage chair.

4. A therapy device as claimed in claim 1 wherein said sensory generating housing is secured to said frame and disposed in close space proximity to the face of the user person, said housing having a closed bottom end, surrounding side walls and an open top end closely spaced to said face of the user person.

5. A therapy device as claimed in claim 4 wherein said closed bottom end is an image generating screen for generating a virtual visual experience or light therapy to the user person.

6. A therapy device as claimed in claim 4 wherein injectors are secured to said bottom or surrounding side walls for injecting ozone, oxygen or a scented odor or a combination of same, into said housing.

7. A therapy device as claimed in claim 6 wherein said injectors are operated as pulsating injectors.

8. A therapy device as claimed in claim 4 wherein a plurality of light generating devices and a sound generating device are secured to one or both of said closed bottom end or surrounding side walls to provide lumino and chromo therapy to the user person.

9. A therapy device as claimed in claim 1 wherein said vibration generating means is constituted by one or more vibrating elements secured to said body supports for imparting a relaxing vibration thereto.

10. A therapy device as claimed in claim 9 wherein said vibrating elements are frequency operated cones secured to said body supports or to frame elements supporting said body supports.

11. A device as claimed in claim 4 wherein said controllable magnetic field generator is positioned and adapted to generate an oriented magnetic field to the body of the user person or part thereof, the intensity, modulation and frequency of said magnetic field being controlled by a selected one of said executable programs.

12. A therapy device as claimed in claim 4 wherein said heating means is comprised by one of electrical heating wires, or flexible heating membrane, secured in said body supports or infrared light generating means, secured to said body supports, to generate heat in specific areas of said body supports, the intensity of said heat being controlled by a selected one of said executable programs.

13. A therapy device as claimed in claim 2 wherein said table is provided with an upper and lower body portion supports, said body supports being pads, each said pads being provided with said body applied therapies, said body applied therapies of said upper and lower body portion support pads being operated independently from one another, said head rest section being provided at a free end of said upper body portion support pad.

14. A therapy device as claimed in claim 3 wherein said massage chair is provided with a seat support, an abdominal support and said head rest support; each said seat and abdominal supports being provided with said body applied therapies, said body applied therapies of said seat and abdominal supports being operated independently from one another.

15. A therapy device as claimed in claim 14 wherein said seat support is secured to a horizontal seat frame portion of said main frame, and an inclined abdominal frame member extending angularly upwards and outwards from an edge section of said horizontal seat frame portion with said abdominal support secured thereto and spaced from said seat support, said head rest support being secured to a free end of said inclined abdominal frame member and spaced from a top end of said abdominal frame.

16. A therapy device as claimed in claim 15 wherein said inclined abdominal frame member is provided with telescopic sections whereby to adjust the spacing between said abdominal support and said seat and said abdominal supports and said head rest support.

17. A therapy device as claimed in claim 1 for use in a personalized therapeutic treatment system comprising a commercially operated spa offering therapeutic services and a household spa having one or more therapeutic treatment devices, an internet link between said commercially operated spa and said household spa to provide PC communication by inputting into a computer information relating to one or more therapy treatment programs assigned to one more domestic user person of said household spa using said treatment device.

18. A therapy device adapted to support the body or part of the body of a user person, said device comprising body supports secured to a support frame and positioned for supporting at least portions of the body of the user person, said body supports having at least one of or a combination of body applied therapies including a vibration generating means, a controllable magnetic field generator and heating means; a controller device having an interface with inputting means for selectively actuating one or all or a combination of said body applied therapies, said controller having a computer and memory for storing executable programs actuable by instructions generated by said inputting means, said body supports being constituted by a seat or a seat portion of a transport vehicle.

19. A therapy device adapted to support a facial part of the body of a user person, said device comprising a head rest support adapted to support a peripheral portion about the face of the user person while permitting exposure to a sensory generating housing having at least one or a combination of facial applied therapies including lumino therapeutic means, an audio therapy device, an aroma therapeutic means; an ozone, oxygen or water vapor injector, and scented essences; a controller device having an interface with inputting means for selecting actuating one or all or a combination of said facial applied therapies, said controller having a computer and memory for storing executable programs actuable by instructions generated by said inputting means, said head rest support being secured to support means disposed forwardly of a seat of a transport vehicle.

20. A personalized therapeutic treatment system comprising a commercially operated spa offering therapeutic services and a household spa having one or more therapeutic treatment devices, an internet link between said commercially operated spa and said household spa to provide PC communication by inputting into a computer information relating to one or more therapy treatment programs assigned to one more domestic user person of said household spa using said treatment device.

21. A personalized therapeutic treatment system as claimed in claim 20 wherein one of said therapeutic treatment devices is a massage table or a massage chair comprising body supports secured to a main frame and positioned for supporting at least portions of the body of the user person, and a head rest support adapted to support a peripheral portion about the face of the user person while permitting face exposure to a sensory generating housing; said sensory generating housing having at least one or a combination of facial applied therapies including a lumino therapeutic means, an audio therapy device, an aroma therapeutic means, an ozone, oxygen or water vapor injector and scented essences; said body supports having at least one or a combination of body applied therapies including a vibration generating means, a controllable magnetic field generator and heating means; and a controller device having an interface with inputting means for selectively actuating each or all or a combination of said facial and body applied therapies, said controller having a computer and memory for storing executable programs actuable by instructions generated by said inputting means.

22. A personalized therapeutic treatment system as claimed in claim 21 wherein said one or more therapy treatment programs comprises massage therapy programs provided by a coach or masseur person of said commercially operated spa at said household spa, said domestic user person providing information pertaining to its treatments to said masseur person through said internet link by said inputting in said computer.

23. A personalized therapeutic treatment system as claimed in claim 20 wherein said therapeutic services comprise the provision of therapy specialists to provide visits to said household spa to effect or assign therapeutic treatments, the monitoring and reporting of communicated information through computers interconnected with said internet link, the provision of therapeutic products personalized to the domestic user person; the provision of personalized classes, courses and techniques relating to therapeutic treatments and products, the provision of internet accessible forms to communicate information on ongoing treatment programs for evaluation of prescribed treatments, and general exercising and nutritional information for the benefit of all domestic user persons clients of the commercial spa.
